(19) United States
(12) Patent Application Publication
(10) Pub. No.: US 2021/0378692 A1
(43) Pub. Date: Dec. 9, 2021

(54) CRANIAL THROMBUS REMOVAL APPARATUS

(71) Applicant: HANGZHOU EXCEED MEDICAL TECHNOLOGY CO., LTD., Hangzhou (CN)

(72) Inventors: Jianping XIANG, Hangzhou (CN); Qimeng ZHOU, Hangzhou (CN); Lingke ZHU, Hangzhou (CN); Hongjuan JING, Hangzhou (CN)

(21) Appl. No.: 17/287,994

(22) PCT Filed: Oct. 22, 2019
(86) PCT No.: PCT/CN2019/112629

§ 371 Date: Apr. 23, 2021

(30) Foreign Application Priority Data
Oct. 23, 2018 (CN) 201811237116.1

(51) Int. Cl. A61B 17/221

(52) U.S. Cl. CPC A61B 17/221; A61B 2017/2212

(57) ABSTRACT

An intracranial thrombus removal apparatus includes a fretwork-type tube-shaped structure, and having a radially compressed loading state and a radially expanded released state. One end of the tube-shaped structure in an axial direction configured for connecting to a delivery is defined as a proximal end, and the other end in the axial direction closed by a mesh cover structure is defined as a distal end a mesh cover structure. The tube-shaped structure comprises a plurality of capturing claws, one end of each capturing claw is defined as a root part connected to a side wall of the tube-shaped structure, and the other end of each of the plurality of capturing claws is defined as a tip extending to an axis position of the tube-shaped structure, and each of the plurality of capturing claws inclines from the proximal end to the distal end while extending.

### CROSS REFERENCE TO RELATED APPLICATIONS

This application is a 371 of international PCT patent application PCT/CN2019/112629 filed on Oct. 22, 2019, which claims all benefits accruing under 35 U.S.C. § 119 from China Patent Application No. 201811237116.1, filed on Oct. 23, 2018, in the China National Intellectual Property Administration, the contents of which are hereby incorporated by reference.

### TECHNICAL FIELD

The present disclosure relates to a medical apparatus, and in particular, to an intracranial thrombus removal apparatus for mechanical thrombectomy.

### BACKGROUND

The main treatment methods for cerebral thrombus are drug thrombolysis and mechanical thrombolysis, but drug thrombolysis therapy has problems including low thrombolysis success rate and narrow time window. In contrast, the time window of mechanical thrombectomy therapy can be extended to 8 hours and the success rate of thrombectomy is higher. Therefore, mechanical thrombectomy has now become the most important method for the treatment of patients with acute ischemic stroke.

The thrombus can be divided into the following types.

1. Pale thrombus. Pale thrombus is mainly composed of many coral-shaped platelet trabecula, and many neutrophils adhere to the surface of the pale thrombus to form a leukoctytic margination. This is presumably attracted by the chemotaxis of cellulose disintegration products. Due to the activated blood coagulation factors, a network of cellulose can be formed between the platelet trabecula, and meshes of the network contain a small amount of blood cells. The pale thrombus is gray-white to the naked eye, with rough and wavy surfaces, hard in quality, and closely connected to the blood vessel wall.

2. Red thrombus is dark red to the naked eye. Fresh red thrombus is moist and elastic. Due to water loss, stale red thrombus becomes dry, fragile and loses elasticity, and is easy to fall off and cause embolism.

3. Mixed thrombus. Mixed thrombus has a structure containing alternately stack-up red stripes and white stripes, or alternately stack-up grey-white strips and rufous strips.

4. Hyaline thrombus. Hyaline thrombus mainly consists of cellulose.

The conventional intracranial thrombus removal apparatus can only capture the red thrombus and the mixed thrombus, but cannot capture the thrombus having complex structures, especially the pale thrombus and the hyaline thrombus, which have relatively long, large or hard structures.

### SUMMARY

In view of this, it is necessary to provide an improved intracranial thrombus removal apparatus, which can improve the effect on thrombectomy of large and hard thrombus.

A intracranial thrombus removal apparatus, includes a fretwork-type tube-shaped structure, which has a radially compressed loading state and a radially expanded released state. One end of the tube-shaped structure in an axial direction is configured for connecting to a delivery device is defined as a proximal end, and the other end of the tube-shaped structure in the axial direction closed by a mesh cover structure is defined as a distal end. The tube-shaped structure includes a plurality of capturing claws. One end of each of the plurality of capturing claws is defined as a root part connected to a side wall of the tube-shaped structure, and the other end of each of the plurality of capturing claws is defined as a tip extending to an axis of the tube-shaped structure. Each of the plurality of capturing claws inclines from the proximal end to the distal end while extending.

A plurality of alternative methods are also provided hereinafter, but they are not intended as additional limitations to the overall scheme, but merely further additions or optimizations. On the premise that there is no technical or logical contradiction, each alternative method can be combined separately with the overall scheme, or a combination of several alternative methods.

In some embodiments, when the tube-shaped structure is in the released state, the tip of each of the plurality of capturing claws freely suspends in a cavity of the tube-shaped structure.

In some embodiments, when the tube-shaped structure is in the released state, remaining parts of each of the plurality of capturing claws freely suspend in the cavity of the tube-shaped structure except the root part.

In some embodiments, the plurality of capturing claws and the tube-shaped structure are an integrity structure formed by a woven or cut process.

In some embodiments, when the tube-shaped structure is in the released state, some or all of the plurality of capturing claws are integrally curved having a recessed portion towards the proximal end.

In some embodiments, each of the plurality of capturing claw includes one rod or a plurality of rods. Each of the plurality of rods includes a distal end and a proximal end, the distal ends of the plurality of rods are near the distal end of the tube-shaped structure, and the proximal end ends of the plurality of rods are near the proximal end of the tube-shaped structure. The plurality of rods are in a furcate shape and distal ends of the plurality of rods are converged together at the tip, and proximal ends of the plurality of rods diverges from one another at the root part and are connected to corresponding positions on the side wall of the tube-shaped structure.

In some embodiments, each of the plurality of capturing claws includes one rod, when the tube-shaped structure is in the released state, an angle between the axis of the tube-shaped structure and a line defined by two ends of the rod is in a range of 10 degrees to 60 degrees.

In some embodiments, each of the plurality of capturing claws includes two rods, and the two rods converge at the tip and form a rounded structure.

In some embodiments, each of the plurality of capturing claws includes two rods. When the tube-shaped structure is in the released state, a line defined by two ends of each of the two rods is regarded as a first datum line. An angle between the two first datum lines corresponding to the two rods is in a range of 30 degrees to 60 degrees.

In some embodiments, each of the plurality of capturing claws includes two rods. When the tube-shaped structure is in the released state, a line defined by two ends of each of the two rods is regarded as a second datum line. The two second datum lines corresponding to the two rods define a datum plane, and an angle between the axis of the tube-shaped structure and the datum plane is in a range of 10 degrees to 60 degrees.

In some embodiments, each of the plurality of capturing claws includes two rods. When the tube-shaped structure is in the released state, a line defined by two ends of each of the two rods is regarded as a second datum line. There is a coplanar bisector between the two second datum lines corresponding to the two rods, and an angle between the bisector and the axis of the tube-shaped structure is in a range of 10 degrees to 60 degrees.

In some embodiments, each of the plurality of capturing claws includes two rods. When the tube-shaped structure is in the released state, a line defined by two ends of each of the two rods is regarded as a second datum line, and the two second datum lines corresponding to the two rods define a datum plane.

A projection of the axis of the tube-shaped structure on the datum plane is a projection line.

An angle between the axis of the tube-shaped structure and the projection line is in a range between 10 degrees to 60 degrees.

In some embodiments, each of the plurality of capturing claws includes two rods. A line defined by a midpoint of the two rods at the root part of each of the plurality of capturing claw and the tip of the capturing claw is regarded as the third datum line. An angle between the axis of the tube-shaped structure and the third datum line is in a range of 10 degrees to 60 degrees.

In some embodiments, the plurality of capturing claws are regarded as one set, the one set of the plurality of capturing claws are disposed along the axis of the tube-shaped structure. A number of the plurality of capturing claws is at least two, and at least two capturing claws are successively disposed along a circumferential direction of the tube-shaped structure. In some embodiments, the plurality of capturing claws are divided into multi sets, which are disposed at intervals along the axis of the tube-shaped structure, and each set of capturing claws includes at least two capturing claws which are successively disposed along the circumferential direction of the tube-shaped structure.

In some embodiments, when the tube-shaped structure is in the releasing state, the tip of the plurality of capturing claws in the same set have a tendency to close to each other.

In some embodiments, adjacent two capturing claws in the same set are parallel to each other along the axis of the tube-shaped structure or interlaced to each other along the axis of the tube-shaped structure.

Each set of capturing claws includes two, four or six capturing claws, which are disposed in staggered arrangement at intervals along the axis of the tube-shaped structure.

Each set of capturing claws includes two to four capturing claws, which are successively disposed in staggered arrangement along the axis of the tube-shaped structure.

In some embodiments, the tube-shaped structure includes a plurality of grid cells. Each of the plurality of grid cells at least includes a first unit and a second unit. An area of the second unit is two to six times of an area of the first unit, and each root part of the plurality of capturing claws is connected to the corresponding second unit.

In some embodiments, when the tube-shaped structure is in the released state, the area of the first unit is in a range of 5 mm² to 10 mm², and the area of the second unit is in a range of 20 mm² to 30 mm².

In some embodiments, at least two adjacent first units are combined with each other and regarded as the second unit.

In some embodiments, four adjacent first units are combined with each other and regarded as the second unit.

In some embodiments, when the tube-shaped structure is in the loading state, and the plurality of capturing claws are accommodated in the second unit connected thereof.

In some embodiments, the second unit has a distal end and a proximal end. The distal end of the second unit is near the distal end of the tube-shaped structure, and the proximal end of the second unit is near the proximal end of the tube-shaped structure. When the tube-shaped structure is in the loading state, the root parts of the plurality of capturing claws are disposed towards the proximal end of the corresponding second unit, and the tips of the plurality of capturing claws extend towards the distal end of the corresponding second unit.

In some embodiments, when the tube-shaped structure is in the loading state, the tips of the plurality of capturing claws extends to the distal end of the corresponding second unit..

In some embodiments, each second unit in the plurality of grid cells is not adjacent therebetween, or at least two second units in the plurality of grid cells are adjacent to each other.

In some embodiments, at least one developing point is disposed at least one of the proximal end, the distal end and a middle portion of the tube-shaped structure in the intracranial thrombus removal apparatus along the axis of the tube-shaped structure.

In some embodiments, the tube-shaped structure includes a plurality of connecting rods, which are gradually gathered together at the proximal end and configured for connecting to the delivery device. A proximal end developing point is disposed at a junction of the plurality of the connecting rods.

In some embodiments, the mesh cover structure includes a distal end and a proximal end. The distal end of the mesh cover structure is near the distal end of the tube-shaped structure, and the proximal end of the mesh cover structure is near the proximal end of the mesh cover structure. The distal end of the mesh cover structure is gathered, and a distal end developing point is disposed at a gathering site of the mesh cover structure get together.

In some embodiments, a middle developing point is disposed at the plurality of capturing claws or the side wall of the tube-shaped structure.

In some embodiments, the developing point is disposed at the tip of some or all of the plurality of capturing claws.

In some embodiments, the developing point on each of the plurality of capturing claw is ring-shaped or stripe-shaped.

In some embodiments, when the tube-shaped structure is in the releasing state, all of the developing points on all of the plurality of capturing claws are arranged along the axis of the tube-shaped structure to define an axis of developing symbols.

In some embodiments, the mesh cover structure has a umbrella shape including a plurality of radially distributed rods; and, the tube-shaped structure includes a plurality of grid cells, a connection portion between proximal ends of the plurality of radially distributed rods and the tube-shaped structure is a vertex of the plurality of grid cells or a junction of adjacent two of the plurality of grid cells, and the proximal ends of the plurality of radially distributed rods are near the proximal end of the tube-shaped structure.

In the present disclosure, capturing claws having suitable extending directions and suitable extending lengths are directly disposed in a thrombus removal instrument, so that the thrombectomy effect is further improved.

### BRIEF DESCRIPTION OF THE DRAWINGS

FIG. 1 is a structural schematic diagram of a intracranial thrombus removal apparatus in one embodiment.

FIG. 2 is a schematic diagram of the intracranial thrombus removal apparatus in a loading state in FIG. 1.

FIG. 3 is a developed view of the intracranial thrombus removal apparatus in FIG. 1.

FIG. 4 is a structural schematic diagram of a intracranial thrombus removal apparatus in another embodiment.

FIG. 5 is a schematic diagram of a partial of the intracranial thrombus removal apparatus in FIG. 4.

FIG. 6a is a developed diagram of the intracranial thrombus removal apparatus in FIG.4.

FIG. 6b is a schematic diagram of a curved tendency of the capturing claw in another embodiment.

FIG. 6c is a schematic diagram of a curved tendency of the capturing claw in another embodiment.

FIG. 7 to FIG. 9 are schematic diagrams the mesh cover structure in the intracranial thrombus removal apparatus from different view angle in another embodiment.

FIG. 10 and FIG. 11 are schematic diagrams the mesh cover structure in the intracranial thrombus removal apparatus from different view angle in another embodiment.

FIG. 12 and FIG. 13a are image development schematic diagrams of a partial of the intracranial thrombus removal apparatus in another embodiment.

FIG. 13b is an image development schematic diagram of the intracranial thrombus removal apparatus in FIG. 13a when it is bent.

FIG. 14 to FIG. 18 are schematic diagrams showing working processes of a intracranial thrombus removal apparatus capturing a normal thrombus in another embodiment.

FIG. 19 is a schematic diagram showing a process of a intracranial thrombus removal apparatus capturing a relatively large thrombus having a complex structure in another embodiment.

FIG. 20 is a schematic diagram showing a process of a intracranial thrombus removal apparatus capturing a fragile thrombus in another embodiment.

FIG. 21 is a structural schematic diagram of a intracranial thrombus removal apparatus in another embodiment.

FIG. 22 to FIG. 26 are developed views of the intracranial thrombus removal apparatus in different embodiments.

FIG. 27 is a developed view of the intracranial thrombus removal apparatus in another embodiment.

### DETAILED DESCRIPTION

The technical solutions in the embodiments of the present disclosure will be clearly and completely described in conjunction with the accompanying drawings in the embodiments of the present disclosure hereinafter. Obviously, the described embodiments are only some of the embodiments of the present disclosure, rather than all the embodiments. Based on the embodiments of the present disclosure, all other embodiments obtained by one of ordinary skill in the art without creative work shall fall within the protection scope of the present disclosure.

In order to better describe and illustrate the embodiments of the present application, one or more drawings can be referred to. However, the additional details or examples used to describe the drawings should not be considered as a limitation on the scope of any of the inventions, the embodiments or preferred modes of the present disclosure.

It should be noted that when a component (or part) is referred to as being “connected” with another component (or part), it can be directly connected to the other component or a central component may also exist. When a component is considered to be “disposed on” another component, it can be directly installed on another component or a centered component may exist at the same time.

Unless otherwise defined, all technical and scientific terms used herein have the same meaning as commonly understood by one of ordinary skill in the technical field of the present disclosure. The terms used in the description of the present disclosure herein are only for the purpose of describing specific embodiments, and are not intended to limit the present disclosure.

Referring to FIG.1, a intracranial thrombus removal apparatus in the present embodiment of the present disclosure can have a fretwork-type tube-shaped structure 1 as a whole. The intracranial thrombus removal apparatus can be made from a metal tube. For example, the intracranial thrombus removal apparatus can be obtained by subjecting a nickel-titanium tube to laser engraving, and then thermoforming. In other embodiments, the intracranial thrombus removal apparatus can be obtained by weaving nickel-titanium wires.

In the present embodiment, the tube-shaped structure 1 of the intracranial thrombus removal apparatus can have a radially compressed loading state and a radially expanded released state. FIG. 2 shows a schematic diagram of the intracranial thrombus removal apparatus in a loading state. When the tube-shaped structure is radially contracted, the tube-shaped structure can have a relatively small external diameter. Therefore, the tube-shaped structure 1 can be conveyed to a focus location through a blood vessel along with a delivery sheath tube. Then the delivery sheath tube can be drawn back, and the tube-shaped structure 1 can expose in the blood vessel, radially expand and be transformed into the released state.

One end of the tube-shaped structure configured for connecting to a delivery device (e.g., a transporting wire) can be defined as a proximal end (e.g., an end at the right side of the tube-shaped structure in FIG. 1). The proximal end can include a plurality of discrete rods, which can converge to form a connecting portion 3. The connecting portion 3 can be connected to the delivery wire, so that the tube-shaped structure 1 can be conveyed in a body and move relative to the delivery sheath tube.

The other end of the tube-shaped structure closed by a mesh cover structure 2 (e.g., an end at the left side of the tube-shaped structure in FIG. 1) can be defined as a distal end mesh cover structure. The mesh cover structure can be umbrella-shaped as a whole, which can diverge from the distal end to the proximal end and be connected to the tube-shaped structure 1, so as to facilitate capturing thrombui with relatively small volumes.

In the present disclosure, the “proximal end” integrally relates to a same direction, and the “distal end” integrally relates to a same direction. When the intracranial thrombus removal apparatus is used, the distal end represents an end near a focus location or a thrombus, and the proximal end is an end away from the focus location or the thrombus. An operator controls the intracranial thrombus removal apparatus from the proximal end.

The tube-shaped structure 1 can have a plurality of capturing claws 4. One end of each of the plurality of capturing claws 4 can be defined as a root part 42 connected to a side wall of the tube-shaped structure 1, and the other end of each of the plurality of capturing claws 4 can be defined as a tip 41 extending to an axis of the tube-shaped structure 1. Each of the plurality of capturing claws 4 inclines from the proximal end to the distal end while extending.

FIG. 1 shows a rough direction along which the capturing claw 4 can extend. When the tube-shaped structure 1 is in the released state, the tip 41 of the capturing claw 4 can extend towards the axis of the tube-shaped structure 1, and can be integrally inclined relative to the axis of the tube-shaped structure. That is, for one capturing claw 4, the tip 41 can be closer to the distal end compared with the root part 42. This can facilitate loading of the tube-shaped structure 1 and the capturing claw 4. When the tube-shaped structure 1 and the capturing claw 4 are loaded into the delivery sheath tube, the capturing claw 4 can gradually be straightened while the tube-shaped structure 1 and the capturing claw 4 entering the sheath tube, and will not become warped and be conveyed to the outside of the sheath tube.

In the intracranial thrombus removal apparatus of the present embodiment, the tube-shaped structure 1 can include a plurality of grid cells, which can have good radial support force and flexibility. A combination of the capturing claw 4 and the mesh cover structure 2 can improve a thrombectomy performance of the intracranial thrombus removal apparatus, especially to thrombus having complex structures.

At least one developing point can be disposed at the proximal end, the distal end and middle of the intracranial thrombus removal apparatus. A material of the developing point can be a platinum-tungsten alloy, a platinum-iridium alloy, a tantalum alloy and the like, but is not limited to this. The developing point can be disposed at any position on the intracranial thrombus removal apparatus, and can be radiopaque.

In another embodiment, a proximal end developing point 5 can be disposed at a proximal end portion of the connecting portion 3. A distal end developing point 6 can be disposed at a distal end portion of the mesh cover structure 2. A middle developing point 7 can be disposed at the tip 41 of the capturing claw 4.

When the tube-shaped structure 1 is in the released state, the tip 41 of each of the capturing claw 4 can freely suspend in a cavity of the tube-shaped structure 1. In some embodiments, when the tube-shaped structure 1 is in the released state, remaining parts of the plurality of capturing claw 4 can freely suspend in the cavity of the tube-shaped structure except the root part 42 of the capturing claw 4.

Referring to FIG. 1 to FIG. 3, the root part 42 of the capturing claw 4 can be fixed to the frame of an adjacent grid cell, and remaining parts of the plurality of capturing claw 4 (as far as the tip) can suspend in the gird cell. When the tube-shaped structure is in the released state, each capturing claw 4 can extend towards/to the axis of the tube-shaped structure 1 and be not limited by other components. That is, the capturing claw 4 can suspend in the cavity of the tube-shaped structure 1. This can facilitate anchoring thrombus having different shapes in the cavity of the tube-shaped structure 1.

In other embodiments, the capturing claw 4 and the tube-shaped structure 1 can be an integrity structure formed by a woven or cut process.

In some embodiments, when the tube-shaped structure 1 is in the released state, some of the capturing claws 4 or all of the capturing claws 4 can be integrally curved having a recessed portion towards the proximal end. That is, while the capturing claw 4 extending towards the distal end from the root part to the tip, the capturing claw 4 can gradually extend towards the axis of the tube-shaped structure along a curved path. An orientation of the recess of the curved structure should be understood as a rough orientation. That is, the curved structure can be arc-shaped or arc-like-shaped, the closer the capturing claw 4 towards the tip is, the faster the capturing claw 4 extends to the axis of the tube-shaped structure.

Some of or all of the capturing claws can have the above curved structure as a whole. It should be understood that at least some of the capturing claws can have a curved structure. In some embodiments, all of the capturing claws can have the curved structure.

The capturing claw in the released state can have a three-dimensional structure. Therefore, the curved structure should be understood as a structural feature of integral the capturing claw. That is, the capturing claw does not extend along a straight line or in a plane, but extends in a curved face or a relatively complex space.

For a capturing claw without the above curved structure, the capturing claw can extend along a straight line or at least extend in a plane. For example, when the tube-shaped structure is in the released state, the capturing claws can be rod-shaped, and extend along a straight line parallel to the rod, respectively. In some embodiments, when the tube-shaped structure is in the released state, the capturing claws can extend in the planes in which it lays, respectively.

In the released state, when the capturing claw 4 extends along a curved path from the root part 42 to the tip 41 and the curved path is an elliptic arc or a similar arc, the circle center of the arc should be at the side near the proximal end of the capturing claw 4. That is, the capturing claw 4 can gradually extend towards the axis of the tube-shaped structure 1 along a curved path. A capturing claw 4, which changes gradually along radial direction of the tube-shaped structure 1, can facilitate capturing thrombus having complex structure.

In some embodiments, the capturing claw 4 can include one rod or a plurality of rods. The plurality of rods can be in a furcate shape and distal ends of the plurality of rods can be converged together at the tip 41, and proximal ends of the plurality of rods can diverge from one another at the root part 42 and be connected to corresponding positions on the side wall of the tube-shaped structure 1.

In some embodiments, each of the plurality of capturing claw can include one rod. When the tube-shaped structure is in the released state, an angle between the axis of the tube-shaped structure and a line defined by two ends of the rod is in a range of 10 degrees to 60 degrees. The rod can be a straight rod or have the above curved structure. All the capturing claws can include straight rods. Alternatively, all the capturing claws can include the above curved structure. Alternatively, some of the capturing claws can include the straight rods, and the other part of the capturing claws can include the above curved claws.

In some embodiments, each of the plurality of capturing claw can include two rods, which can be a rod 43 and a rod 44, respectively. The rod 43 and the rod 44 can converge at the tip 41 of the capturing claw 4. In order to improve security of the capturing claw 4, in some embodiments, the rod 43 and the rod 44 can form a rounded structure.

In some embodiments, each of the plurality of capturing claw can include two rods, which can be a rod 43 and a rod 44, respectively. The rod 43 and the rod 44 can converge at the tip 41 of the capturing claw 4. When the tube-shaped structure is in the released state, a line defined by two ends of each of the two rods can be regarded as a first datum line. An angle between the two first datum lines corresponding to the two rods can be in a range of 30 degrees to 60 degrees. The angle between the two datum lines can affect the approaching between the two rods. The larger the angle is, the easier the two rods can approach to each other.

On the contrary, the smaller the angle is, the harder the two rods can approach to each other, and the longer and the narrower the capturing claw 4 as a whole is. Therefore, the angle between the two first datum lines can also influence the effect for capturing the thrombus. The range provided in the present embodiment can facilitate improving the effect for capturing the thrombus.

Referring to FIG. 4 to FIG. 6a, in some embodiments, less grid cells can be arranged along the width direction in the developed view. Three capturing claws 4 can be arranged along the width direction, so that a corresponding tube-shaped structure can have a smaller diameter. Compared with the embodiment in FIG.4 to FIG. 6a, four capturing claws 4 are arranged along a width direction in FIG. 1 to FIG. 4, and the tube-shaped structure in FIG. 1 to FIG. 4 can have a larger diameter.

In the present embodiment, in the released state, the capturing claw 4 can have a curved structure as whole. That is, while the capturing claw extending towards the distal end from the root part to the tip, the capturing claw can gradually extend towards the axis of the tube-shaped structure along a curved path. A recess of the curved structure faces towards the proximal end. The curved structure can be arc-shaped or arc-like-shaped. The closer the capturing claw towards the tip is, the faster the capturing claw extends to the axis of the tube-shaped structure.

In FIG. 4 to FIG. 6a, each of the capturing claws 4 can include two rods, which can be rod 45 and rod 46, respectively. When the tube-shaped structure is in the released state, a line defined by two ends of each of the two rods can be regarded as a second datum line. There can be a coplanar bisector 47 between the two second datum lines corresponding to the two rods, and an angle between the bisector 47 and the axis L of the tube-shaped structure 1 can be in a range of 10 degrees to 60 degrees. In some embodiments, the angle can be in a range of 30 degrees to 60 degrees.

The angle between the bisector 47 and the axis L of the tube-shaped structure 1 can determine a tendency that the capturing claw 4 inclines and bends, and can further influence the performance of the capturing claws for capturing the thrombus. The range provided in the present embodiment can facilitate improving the effect for capturing the thrombus.

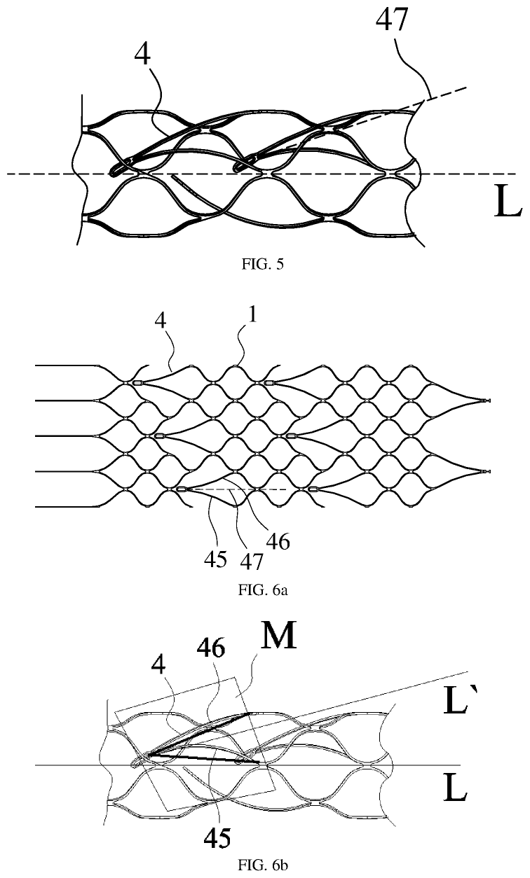

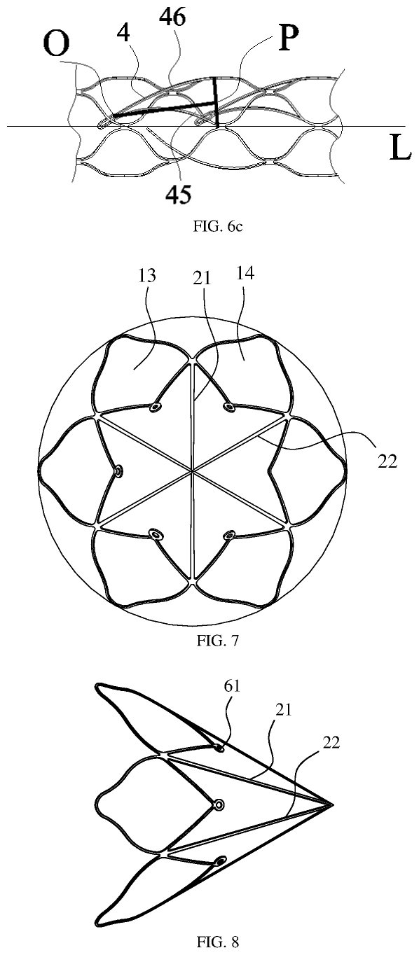

Referring to FIG. 6b, in some embodiments, each of the capturing claws 4 can include two rods, that is, a rod 45 and a rod 46, respectively. When the tube-shaped structure 1 is in the released state, a line defined by two ends of each of the two rods can be regarded as a second datum line, and the two second datum lines corresponding to the two rods (the two heavy lines in FIG. 6b) can define a datum plane M. An angle between the axis L of the tube-shaped structure and the datum plane M can be in a range of 10 degrees to 60 degrees. Alternatively, a projection of the axis L of the tube-shaped structure on the datum plane M can be a projection line L′. An angle between the axis 1 of the tube-shaped structure and the projection line L′ can be in a range of 10 degrees to 60 degrees.

The angle between the axis L of the tube-shaped structure 1 and the projection line L′ can determine a tendency that the capturing claw 4 inclines and bends, and can further influence the performance of the capturing claws for capturing the thrombus. The range provided in the present embodiment can facilitate improving the effect for capturing the thrombus.

Referring to 6c, in some embodiments, each of the capturing claws 4 can include two rods, that is a rod 45 and a rod 46, respectively. When the tube-shaped structure 1 is in the released state, a line defined by a midpoint P of the two rods at the root part of the capturing claw and the tip O of the capturing claw can be regarded as the third datum line OP. An angle between the axis L of the tube-shaped structure and the third datum line OP can be in a range of 10 degrees to 60 degrees.

The angle between the axis L of the tube-shaped structure 1 and the third datum line OP can determine a tendency that the capturing claw 4 inclines and bends, and can further influence the performance of the capturing claws for capturing the thrombus. The range provided in the present embodiment can facilitate improving the effect for capturing the thrombus.

The plurality of capturing claw 4 can define a three-dimensional space for inserting the thrombus. Adjacent rods can form dense grid cells on a radial section of the tube-shaped structure 1, and the dens grid cells can effectively intercept a fallen-off thrombus.

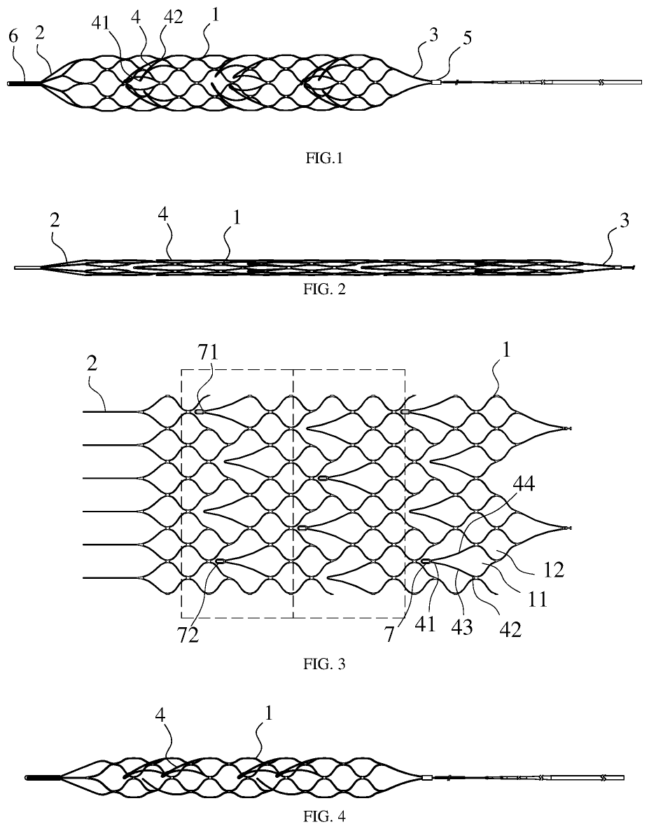

In some embodiments, the plurality of capturing claws can be regarded as one set, or the plurality of capturing claws can be divided into multi sets, which are disposed at intervals along the axis of the tube-shaped structure. For example, in FIG. 3, the capturing claws in each dotted box can be regarded as one set, and there can be three sets of capturing claws as a whole. In FIG. 6a, there can be two sets of capturing claws as a whole.

One set of the capturing claws can include at least two capturing claws which can be successively disposed along the circumference of the tube-shaped structure. For example, in FIG. 3, there can be four capturing claws in one set. In FIG. 5, there can be three capturing claws in one set. In one set of the capturing claws, adjacent two capturing claws can be parallel to each other along the axis of the tube-shaped structure or interlaced to each other along the axis of the tube-shaped structure.

When the tube-shaped structure is in the released state, the tips of the capturing claws in each set can be gathered. Therefore, the capturing claws can effectively intercept the thrombus, and define the three-dimensional space for inserting the thrombus with the fretwork-type tube-shaped structure 1, so as to largely improve fastness for fixing the thrombus.

In some embodiments, each set of the capturing claws can include two, four or eight capturing claws, which can be in staggered arrangement at intervals along the axis of the tube-shaped structure.

In some embodiments, each set of the capturing claws includes two to four capturing claws, which can be successively disposed in staggered arrangement along the axis of the tube-shaped structure.

In some embodiments, the tube-shaped structure 1 can include a plurality of grid cells. For example, in FIG. 3, the grid cell can at least include a first unit 12 and a second unit 11. An area of the second unit 11 can be two to six times of an area of the first unit 12 (e.g., four times), and the root part 42 of the capturing claw can be connected to the corresponding second unit.

In some embodiments, the grid cell can have a closed loop structure, which can have relatively strong radial support force. In the present embodiment, the second unit 11 having a larger area can be provided, so that the tube-shaped structure 1 can integrally have relatively good flexibility.

The second unit 11 having a larger area can facilitate the thrombus with a larger volume entering the tube-shaped structure 1. For example, in FIG. 3, each capturing claw 4 can include two rods, that is, a rod 43 and a rod 44. A proximal end of the rod 44 can be connected to a vertex of the second unit 11. The rod 43 can be provided in a similar way. The capturing claw can be disposed at the second unit 11 having the relative large area. The capturing claw and the second unit 11 can supplement with each other, so as to facilitate capturing the thrombus having the complex structure.

The grid cell can be quadrangular or hexagonal, but is not limited to this. In the present embodiment, the fretwork-type tube-shaped structure 1 can be consisted of two kinds of closed grid cells having different areas. The first unit 12 having the smaller area can provide relatively large radial support force, and the second unit 11 having the larger area can facilitate capturing thrombus having relative large size and complex structure, and reducing a contact surface between the tube-shaped structure and the vascular wall. The larger grid cell and the smaller grid cell can be disposed at intervals, so that the cerebral thrombus removal instrument can have excellent radial support force and thrombus capturing performance

When the tube-shaped structure 1 is in the released state (as shown in a developed view), the area of the first unit can be in a range of 5 mm² to 10 mm², and the area of the second unit can be in a range of 20 mm² to 30 mm².

In some embodiments, at least two adjacent first units 12 can be combined with each other to define one corresponding second unit 11. For example, in FIG. 3 and FIG. 6, four adjacent first units can be combined with each other, defining one corresponding second unit.

Referring to FIG. 2 and FIG. 3, when the tube-shaped structure is in the loading state, the capturing claw 4 can be accommodated in the second unit 11 connected thereof. The root part 42 of the capturing claw 4 can be disposed at a proximal end portion of the second cell 11, and the tip 41 of the capturing claw 4 extends towards a distal end portion of the second unit 11.

In order to form a relatively long capturing claw 4, when the tube-shaped structure is in the loading state, the tip 41 of the capturing claw 4 can extend to a distal end portion of the second unit 11. That is, the capturing claw 4 should fill the second unit 11 along the axis of the tube-shaped structure as far as possible. There can be a plurality of second units 11, and the second units cannot be adjacent to each other, or at least two second units can be adjacent to each other. In the present embodiment, the second units corresponding to the capturing claws in the same set can be successively adjacent to each other along a circumference of the tube-shaped structure.

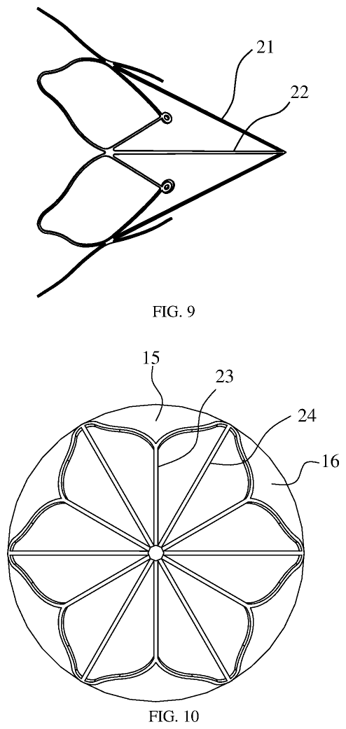

Referring to FIG. 7 to FIG. 9, in some embodiments, the mesh cover structure can have a structure center at a side near the distal end of the intracranial thrombus removal apparatus. The mesh cover structure can radially divaricate from the structure center to the proximal end to form an umbrella-shaped open end. The umbrella-shaped open end can be connected to the distal end of the tube-shaped structure. The mesh cover structure can prevent the thrombus from peeling off or escaping, so as to effectively avoid a second thrombosis. The structure center can be located on the axis of the tube-shaped structure, so that the umbrella-shaped structure can be more symmetrical and facilitate loading.

The tube-shaped structure 1 can include a plurality of grid cells near the distal end. A first unit 13 and a first unit 14 can be seen in the figures. The umbrella-shaped structure can include a plurality of radially distributed rods, which extend from the distal end to the proximal end. The mesh cover structure can include six rods, for example, a rod 21 and a rod 22 in the figures.

A junction of the proximal end of the rod and the tube-shaped structure can be a vertex of the grid cell or a junction of two adjacent grid cells. In FIG. 7, a rod 21 can be connected to a junction of the first unit 13 and the first unit 14.

The distal end of the mesh cover structure or the tube-shaped structure can be provided with a developing point. For example, the developing point 61 can be provided at the vertex of the distal end of the first unit. The vertexes of the distal end of the first unit are further provided with developing points.

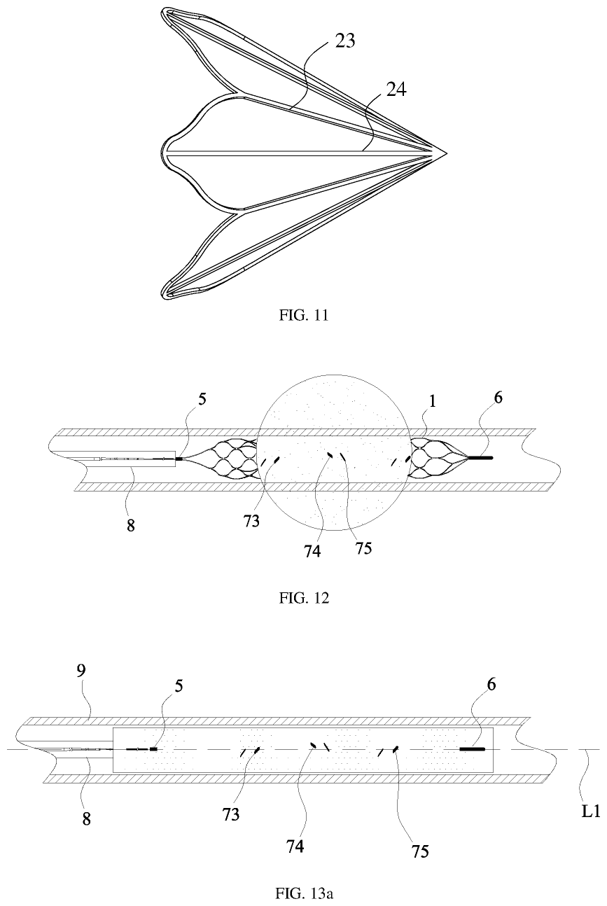

Referring to FIG. 10 and FIG. 11, in some embodiments, compared with the embodiments above, the mesh cover structure can include twelve rods, so that the mesh cover structure can be denser. The tube-shaped structure 1 can include a plurality of grid cells near the distal end. A first unit 15 and a first unit 16 can be seen in the figures. In the figures, a rod 23 can be connected to the vertex of the first unit 15, and a rod 24 can be connected to a junction of the first unit 15 and the first unit 16.

In general, the mesh cover structure can be consisted of a plurality of radially disposed rods; alternatively, can be consisted of underlaps of the smaller grid cells. A number of the rods can be in a range of four to twelve. In released state, an angle between each rod and the axis of the tube-shaped structure can be in range of 15 degrees to 45 degrees. Grid cells having suitable sizes can prevent the thrombus from peeling off or escaping.

The denser the mesh cover structure is, the harder the thrombus can be peeled off and escaped. The number of the rods and the angle between the rod and the axis of the tube-shaped structure can influence the density. The range provided in the present embodiment can give consideration to transporting performance and density of the intracranial thrombus removal apparatus, so that the intracranial thrombus removal apparatus can capture the fragile thrombus, e.g., a red thrombus.

At least one developing point can be disposed at least one of the proximal end, the distal end and a middle portion of the tube-shaped structure in the intracranial thrombus removal apparatus along the axis of the tube-shaped structure. The developing point can be made from metal material such as a platinum-tungsten alloy, a platinum-iridium alloy, a tantalum alloy and the like, but is not limited to this. The developing point can be disposed at any position on the intracranial thrombus removal apparatus, and can be radiopaque.

Referring to FIG. 1, in some embodiments, the connecting portion 3 can include a plurality of gradually gathered connecting rods configured for connecting to a delivery device. A proximal end developing point 5 can be disposed at a junction of the plurality of the connecting rods. The proximal end developing point 5 can be connected to a junction of the intracranial thrombus removal apparatus and the delivery wire via a developing ring or a spring by method of welding.

The distal end of the mesh cover structure 2 can be gathered, and a distal end developing point 6 can be disposed at a gathering site of the mesh cover structure get together. The distal end developing point 6 can gather the rods of via a spring by method of welding.

A middle developing point 7 can be disposed at the tip 41 of some of the capturing claws 4, for example, a middle developing point 71 and a middle developing pint 72 in FIG. 3. The middle developing point can be ring-shaped or stripe-shaped, and can be fixed to the tip of the corresponding capturing claw by methods of welding or riveting.

In some embodiments, the intracranial thrombus removal apparatus can include the near-end developing points 5, the distal end developing point 6 and the middle developing points, that is, a plurality of developing points can be disposed throughout the intracranial thrombus removal apparatus. Therefore, the intracranial thrombus removal apparatus can be integrally radiopaque.

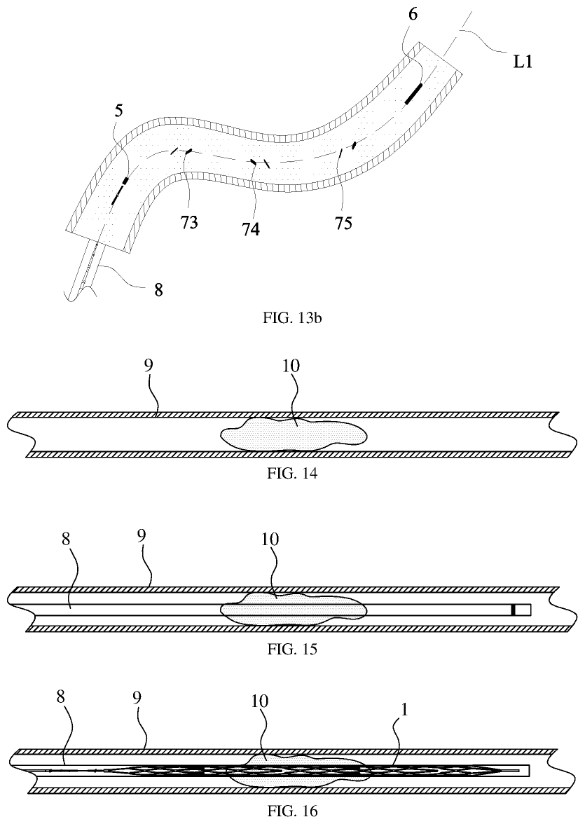

Referring to FIG. 12, a developing effect is shown in a round frame. Referring to FIG. 13a, a developing effect is shown in a square frame. In a blood vessel 9, after the delivery sheath tube 8 is drawn back, the tube-shaped structure 1 of the intracranial thrombus can be released. An end of the delivery sheath tube 8 can further be provided with a developing point. A combination of the developing point on the delivery sheath tube 8, the near-end developing points 5, the distal end developing point 6 and the plurality of the middle developing points can provide good developing performance to the intracranial thrombus removal apparatus.

In some embodiments, when the tube-shaped structure is in the releasing state, all of the developing points on all of the plurality of capturing claws can be arranged along the axis of the tube-shaped structure to define an axis L1 of developing symbols.

Referring to FIG. 12, FIG. 13a, and FIG. 13b, the plurality of capturing claws 4 can be divided into three sets, which are disposed along the axis of the tube-shaped structure. The developing point is disposed at the tip of two of the capturing claws in each set. All of the developing points on each capturing claw can be arranged along the axis of the tube-shaped structure to define an axis of developing symbols. Taking the middle developing point 73, the middle developing point 74 and the middle developing point 75 in the figures as an example, the middle developing points can be roughly disposed along the axis of the tube-shaped structure, and can show the integral position of the tube-shaped structure. Since all of the middle developing points are disposed on the axis of the tube-shaped structure, radial edges of the tube-shaped structure and the relative position of the tube-shaped structure in the blood vessel can be indirectly predicted. If the developing points are only disposed at side walls of the tube-shaped structure 1, there will be relatively large deviations from different angle of views. In the present embodiment, all of the developing points on the tube-shaped structure can define an axis L1 of developing symbols. Therefore, even when the intracranial thrombus removal apparatus is curved as shown in FIG. 13b, the axis L1 of developing symbols can accurately show the axis of the tube-shape structure.

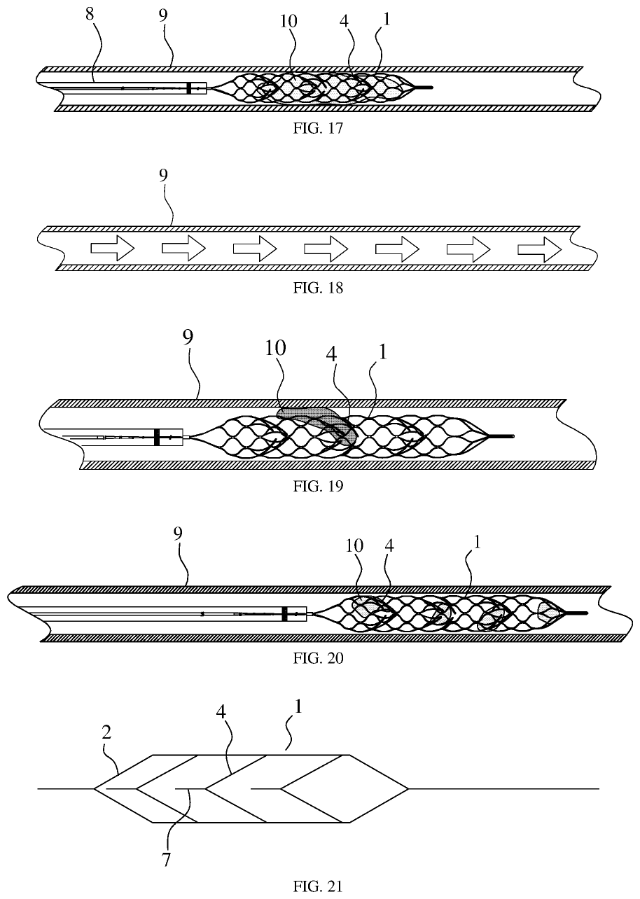

Referring to FIG. 14 to FIG. 18, the process for capturing a thrombus with the intracranial thrombus removal apparatus in some embodiments can be shown hereinafter. There can be a thrombus 10 in a blood vessel 9. A delivery sheath tube 8 can carry the intracranial thrombus removal apparatus through the thrombus region, and then the delivery sheath tube 8 can be drawn back, so that the intracranial thrombus removal apparatus can be released, i.e., the tube-shaped structure radially expand. Therefore, the thrombus 10 can gradually enter a cavity of the tube-shaped structure 1, and is anchored by the plurality of capturing claws 4. Finally, the intracranial thrombus removal apparatus can be drawn back, and the thrombus 10 can be taken out by the capturing claw 4 and the mesh cover structure at the distal end. Therefore, the blood flow can be unobstructed again.

Referring to FIG. 19, in some embodiments, when the intracranial thrombus removal apparatus captures a thrombus having a complex structure and a relatively large size and the tube-shaped structure 1 radially expands, the relatively large thrombus 10 can enter the cavity of the tube-shaped structure 1 through a grid cell having a relatively large area and be anchored by capturing claws 4 at corresponding positions. If all of the grid cells are the first units having a small area, the relatively large thrombus 11 can be difficult to enter the cavity of the tube-shaped structure 1, and difficult to be taken out. In view of this, the intracranial thrombus removal apparatus in the present embodiment can capture the thrombus having complex structure.

Referring to FIG. 20, in some embodiments, when the intracranial thrombus removal apparatus captures a fragile thrombus, the fragile thrombus can enter the cavity of the tube-shaped structure 1 and be anchored by contracted capturing claws 4 when the tube-shaped structure 1 radially expands. The capturing claw 4 and a dense mesh cover structure can cooperate with each other at the distal end, and capture and take out the thrombus. In view of this, the intracranial thrombus removal apparatus in the present embodiment can capture the thrombus having complex structure.

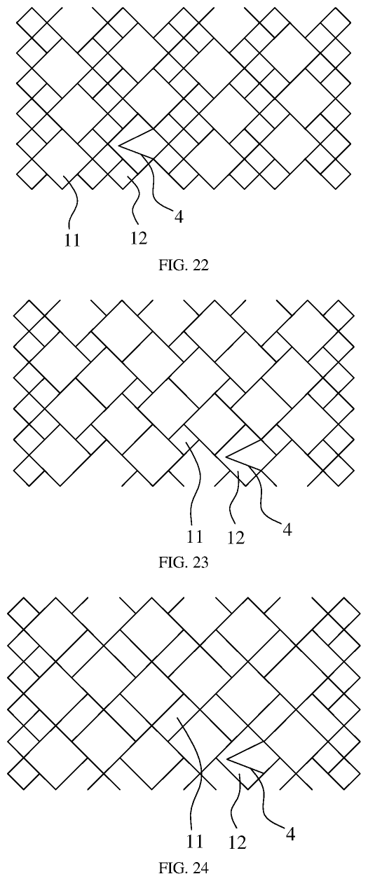

Referring to FIG. 21, in some embodiments, the distal end of the tube-shaped structure 1 can have a mesh cover structure 2, and the middle developing point 7 can be disposed on the capturing claw 4 of the tube-shaped structure 1. The other structural features of the intracranial thrombus removal apparatus in the present disclosure can be or refer to those in the above embodiments.

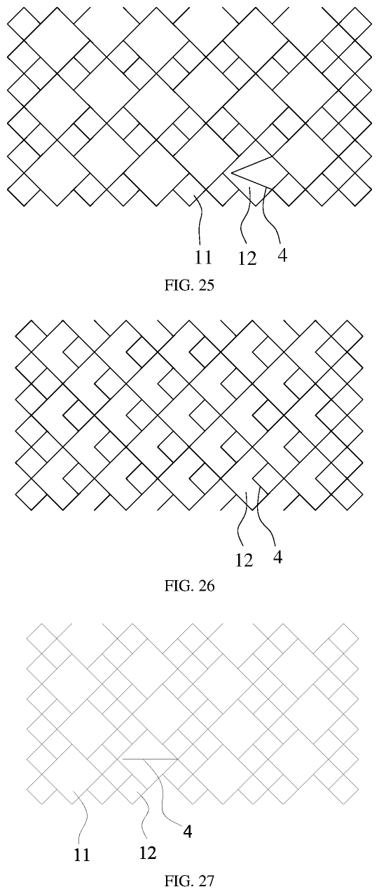

Referring to FIG. 22 to FIG. 26, in some embodiments, shapes and distributions of the second unit 11 and the first unit 12 can be slightly different from those in the above embodiments. The other structural features of the intracranial thrombus removal apparatus in the present disclosure can be or refer to those in the above embodiments. In the figures, the capturing claw 4 can include two rods, which can be fixed to the corresponding second unit 11. In the embodiments, each second unit 11 can be provided with the capturing claw 4. In the figures, the capturing claw 4 is only shown in one of the second units 11, and the other second units 11 can be provided in a similar way.

Referring to FIG. 27, compared with the embodiments above, the intracranial thrombus removal apparatus in the present embodiment can have following differences. The capturing claw 4 can have a single rod. A shape and distribution of the second unit 11 and the first unit 12 can be different. The other structural features of the intracranial thrombus removal apparatus in the present disclosure can be or refer to those in the above embodiments. When a tube-shaped structure 1 of the present embodiment is in the released state, a distal end of the capturing claw 4 can gradually extend to an axis of the tube-shaped structure along a straight line. In some embodiments, the capturing claw 4 can have a curved structure. For example, the capturing claw 4 can extend to the axis of the tube-shaped structure along an arc path or an arc-like path.

The technical features of the above-mentioned embodiments can be combined arbitrarily. In order to make the description concise, all possible combinations of the various technical features in the foregoing embodiments are not described. However, as long as there is no contradiction in the combination of these technical features, it should be regarded as the scope described in this specification.

The above-mentioned embodiments only express several embodiments of the present disclosure, and the descriptions are relatively specific and detailed, but they should not be interpreted as limiting the scope of the disclosure patent. It should be pointed out that for one of ordinary skill in the art, without departing from the concept of the present disclosure, several modifications and improvements can be made, and these all fall within the protection scope of the present disclosure. Therefore, the scope of protection of the present disclosure shall be subject to the appended claims.

1. An intracranial thrombus removal apparatus, comprising a fretwork-type tube-shaped structure, which has a radially compressed loading state and a radially expanded released state,
one end of the tube-shaped structure in an axial direction configured for connecting to a delivery device is defined as a proximal end, and the other end of the tube-shaped structure in the axial direction closed by a mesh cover structure is defined as a distal end,
wherein the tube-shaped structure comprises a plurality of capturing claws, one end of each of the plurality of capturing claws is defined as a root part connected to a side wall of the tube-shaped structure, the other end of each of the plurality of capturing claws is defined as a tip extending to an axis of the tube-shaped structure, and each of the plurality of capturing claws is inclined from the proximal end to the distal end.

2. The intracranial thrombus removal apparatus of claim 1, wherein when the tube-shaped structure is in the released state, the tip of each of the plurality of capturing claws freely suspends in a cavity of the tube-shaped structure; or, when the tube-shaped structure is in the released state, remaining parts of each of the plurality of capturing claws freely suspend in the cavity of the tube-shaped structure except the root part.

3. (canceled)

4. The intracranial thrombus removal apparatus of claim 1, wherein the plurality of capturing claws and the tube-shaped structure are an integrity structure.

5. The intracranial thrombus removal apparatus of claim 1, wherein when the tube-shaped structure is in the released state, some or all of the plurality of capturing claws are integrally curved having a recessed portion towards the proximal end.

6. The intracranial thrombus removal apparatus of claim 1, wherein each of the plurality of capturing claw comprises one rod, when the tube-shaped structure is in the released state, an angle between the axis of the tube-shaped structure and a line defined by two ends of the rod is in a range of 10 degrees to 60 degrees.

7. (canceled)

8. The intracranial thrombus removal apparatus of claim 1, wherein each of the plurality of capturing claw comprises a plurality of rods, and each of the plurality of rods comprises a distal end and a proximal end, the distal ends of the plurality of rods are near the distal end of the tube-shaped structure, and the proximal ends of the plurality of rods are near the proximal end of the tube-shaped structure; and
the plurality of rods are in a furcate shape and distal ends of the plurality of rods are converged together at the tip, and proximal ends of the plurality of rods diverges from one another at the root part and are connected to corresponding positions on the side wall of the tube-shaped structure

9. The intracranial thrombus removal apparatus of claim 6, wherein each of the plurality of capturing claw comprises two rods, the two rods converge at the tip and form a rounded structure; or,
when the tube-shaped structure is in the released state, a line defined by two ends of each of the two rods is regarded as a first datum line, and an angle between the two first datum lines corresponding to the two rods is in a range of 30 degrees to 60 degrees; or,
when the tube-shaped structure is in the released state, the line defined by two ends of each of the two rods is regarded as a second datum line, the two second datum lines corresponding to the two rods define a datum plane, and an angle between the axis of the tube-shaped structure and the datum plane is in a range of 10 degrees to 60 degrees .

10. (canceled)

11. The intracranial thrombus removal apparatus of claim 1, wherein the plurality of capturing claws are regarded as one set, the one set of the plurality of capturing claws are disposed along the axis of the tube-shaped structure, a number of the plurality of capturing claws is at least two, and at least two capturing claws are successively disposed along a circumferential direction of the tube-shaped structure; or
the plurality of capturing claws are divided into multi sets, which are disposed at intervals along the axis of the tube-shaped structure, and each set of capturing claws comprises at least two capturing claws which are successively disposed along the circumferential direction of the tube-shaped structure; or,
adjacent two capturing claws in the same set are parallel to each other along the axis of the tube-shaped structure or interlaced to each other along the axis of the tube-shaped structure; or,
each set of capturing claws comprises two, four or six capturing claws, which are disposed in staggered arrangement at intervals along the axis of the tube-shaped structure; or,
each set of capturing claws comprises two to four capturing claws, which are successively disposed in staggered arrangement along the axis of the tube-shaped structure.

12. The intracranial thrombus removal apparatus of claim 11, wherein when the tube-shaped structure is in the releasing state, the tip of the plurality of capturing claws in the same set have a tendency to close to each other.

13-15. (canceled)

16. The intracranial thrombus removal apparatus of claim 1, wherein the tube-shaped structure comprises a plurality of grid cells, each of the plurality of grid cells at least comprises a first unit and a second unit, and an area of the second unit is two to six times of an area of the first unit, and each root part of the plurality of capturing claws is connected to the corresponding second unit.

17. The intracranial thrombus removal apparatus of claim 16, wherein when the tube-shaped structure is in the released state, the area of the first unit is in a range of 5 mm² to 10 mm², and the area of the second unit is in a range of 20 mm² to 30 mm².

18. The intracranial thrombus removal apparatus of claim 16, wherein at least two adjacent first units are combined with each other or,
four adjacent first units are combined.

19. (canceled)

20. The intracranial thrombus removal apparatus of claim 16, wherein when the tube-shaped structure is in the loading state, the plurality of capturing claws are accommodated in the second unit connected thereof.

21. The intracranial thrombus removal apparatus of claim 20, wherein
the second unit has a distal end and a proximal end, the distal end of the second unit is near the distal end of the tube-shaped structure, and the proximal end of the second unit is near the proximal end of the tube-shaped structure,
when the tube-shaped structure is in the loading state, the root parts of the plurality of capturing claws are disposed towards the proximal end of the corresponding second unit, and the tips of the plurality of capturing claws extend towards the distal end of the corresponding second unit;
or, when the tube-shaped structure is in the loading state, the tips of the plurality of capturing claws extends to the distal end of the corresponding second unit.

22. (canceled)

23. The intracranial thrombus removal apparatus of claim 16, wherein each second unit in the plurality of grid cells is not adjacent therebetween, or at least two second units in the plurality of grid cells are adjacent to each other.

24. The intracranial thrombus removal apparatus of claim 1, wherein at least one developing point is disposed at least one of the proximal end, the distal end and a middle portion of the tube-shaped structure in the intracranial thrombus removal apparatus along the axis of the tube-shaped structure; or,
the tube-shaped structure comprises a plurality of connecting rods, which are gradually gathered together at the proximal end and configured for connecting to the delivery device, and a proximal end developing point is disposed at a junction of the plurality of the connecting rods; or,
the mesh cover structure comprises a distal end and a proximal end, the distal end of the mesh cover structure is near the distal end of the tube-shaped structure, and the proximal end of the mesh cover structure is near the proximal end of the tube-shaped structure,
the distal end of the mesh cover structure is gathered, and a distal end developing point is disposed at a gathering site of the mesh cover structure get together; or
a middle developing point is disposed at the plurality of capturing claws or the side wall of the tube-shaped structure.

25-27. (canceled)

28. The intracranial thrombus removal apparatus of claim 24, wherein the developing point is disposed at the tip of some or all of the plurality of capturing claws.

29. The intracranial thrombus removal apparatus of claim 24, wherein when the tube-shaped structure is in the releasing state, all of the developing points on all of the plurality of capturing claws are arranged along the axis of the tube-shaped structure to define an axis of developing symbols.

30. The intracranial thrombus removal apparatus of claim 16, wherein the distal end of the mesh cover structure in the intracranial thrombus removal apparatus is gathered near the axis of the tube-shaped structure.

31. The intracranial thrombus removal apparatus of claim 30, wherein the mesh cover structure having a umbrella shape comprises a plurality of radially distributed rods, the tube-shaped structure comprises a plurality of grid cells, a connection portion between proximal ends of the plurality of radially distributed rods and the tube-shaped structure is a vertex of the plurality of grid cells or a junction of adjacent two of the plurality of grid cells, and the proximal ends of the plurality of radially distributed rods are near the proximal end of the tube-shaped structure.
